(19) United States
(12) Patent Application Publication
(10) Pub. No.: US 2011/0118650 A1
(43) Pub. Date: May 19, 2011

(54) METHOD AND APPARATUS FOR TREATING OBESITY AND CONTROLLING WEIGHT GAIN AND ABSORPTION OF GLUCOSE IN MAMMALS

(75) Inventor: Raj Nihalani, Irvine, CA (US)

(73) Assignee: Anteromed, Inc., Irvine, CA (US)

(21) Appl. No.: 12/621,443

(22) Filed: Nov. 18, 2009

(51) Int. Cl. A61F 2/04; A61M 25/00; A61B 17/12

(52) U.S. Cl. 604/9; 604/8; 606/151

(57) ABSTRACT

The present invention includes an endoluminal sleeve having a stomach sleeve portion, an antral sleeve portion, and an intestine sleeve portion. The endoluminal sleeve includes a flexible core layer which is formed from a self-expanding material. The flexible core layer in the stomach sleeve portion can self expand to grip the stomach. The antral sleeve is located in the pyloric antrum and is connected to the stomach sleeve portion and the intestine sleeve portion. The intestine sleeve portion is connected to the antral sleeve through a junction. The junction contains non-self-expanding material allowing the pyloric sphincter to properly function. The intestine sleeve portion is located in the small intestines. The intestine sleeve portion can have fenestrations, roots, a nutrient delivery layer, a nutrient delivery cover, an outer shell with a plurality of holes, and/or a semi-porous skin for nutrient delivery to the small intestines.

### BACKGROUND

1. Field

The invention relates to a method and apparatus for treating obesity and controlling weight gain and absorption of glucose and fat in mammals, and more specifically, to using an endoluminal sleeve for treating obesity and controlling weight gain and absorption of glucose in mammals.

2. Related Art

Extreme obesity is a major illness in the United States and other developed countries. More than half of Americans are overweight, while nearly one-third are categorized as obese. Obesity is the accumulation of excess fat on the body, and is defined as having a body mass index (BMI) of greater than 30. Many serious long-term health consequences are associated with obesity, such as, hypertension, diabetes, coronary artery disease, stroke, congestive heart failure, venous disease, multiple orthopedic problems and pulmonary insufficiency with markedly decreased life expectancy.

Medical management of obesity including dietary, psychotherapy, medications and behavioral modification techniques have yielded extremely poor results in terms of treating obesity. Several surgical procedures have been tried which have bypassed the absorptive surface of the small intestine or have been aimed at reducing the stomach size by either partition or bypass. These procedures have been proven both hazardous to perform in morbidly obese patients and have been fraught with numerous life-threatening postoperative complications. Moreover, such operative procedures are often difficult to reverse.

One procedure for treating morbid obesity is referred to as vertical sleeve gastrectomy (“sleeve gastrectomy”). In a sleeve gastrectomy procedure, the stomach is laparoscopically reduced to about 15% of its original size by surgical removal of a large portion of the stomach, following the greater curvature. The open edges are then attached together (often with surgical staples) to form a sleeve or tube with a banana shape. The procedure permanently reduces the size of the stomach, and is not reversible. Because a part of the stomach is cut and secured with the help of a stapler in this procedure, the operation can be risky. A few risks, for example, are bleeding and leakage of stomach contents if the staples are not secure, or open up. This leakage can be life threatening and difficult to heal.

In other bariatric procedures, portions of the stomach and small intestines are bypassed, causing minimally digested or undigested food to be deposited far into the intestines, where the jejunum is stimulated from exposure to undigested food. However, these procedures block or severely limit the amount of essential nutrients that get absorbed by the body through the small intestines. Furthermore, conventional bariatric procedures that insert material into the pyloric sphincter can reduce the ability of the pyloric sphincter to contract and relax efficiently, resulting in difficulty in emptying the stomach. This can also create a potentially untoward adverse condition for the patient.

Therefore, there is a need for a method and apparatus for treating obesity and controlling weight gain and glucose and fat absorption in mammals that reduce leakage in the stomach, maintain the pyloric sphincter in a properly functioning state, and allow a proper amount of essential nutrients to get absorbed by the body.

### SUMMARY

The present invention is directed to a method and apparatus for treating obesity and controlling weight gain and glucose and fat absorption in mammals, the method and apparatus reduce leakage in the stomach, allow the pyloric sphincter to function properly, and improve the amount of essential nutrients that get absorbed by the body. The present invention uses an endoluminal sleeve in a stomach that has undergone sleeve gastrectomy and an intestine in order to bypass the stomach and the intestine. The endoluminal sleeve includes a stomach sleeve portion, an antral sleeve portion, and an intestine sleeve portion. The endoluminal sleeve can include a flexible core layer which is formed from a self-expanding material.

The stomach sleeve portion is placed in the stomach, and the flexible core layer can self expand to allow the stomach sleeve portion to grip the stomach. In the case of a gastric sleeve procedure, the stomach sleeve portion prevents the acids, pressure, and/or contents in the stomach from interfering with the staple line in the stomach. This reduces the possibility of a bleeding and/or a leakage in the stomach. The antral sleeve is located in the pyloric antrum and is connected to both the stomach sleeve portion and the intestine sleeve portion. The intestine sleeve portion is connected to the antral sleeve through a junction. The junction can be filled with non-self-expanding material to maintain the pyloric sphincter in a properly functioning state to empty stomach contents. In another embodiment, the junction can be devoid of material to maintain the pyloric sphincter in a properly functioning state to empty stomach contents.

The intestine sleeve portion can be located in the intestines prior to the jejunum. The intestine sleeve portion can have fenestrations and/or roots. The fenestrations and/or roots can allow nutrients flowing through the intestine sleeve portion to reach the small intestine. Furthermore, the roots can also provide a gripping function to prevent the intestine sleeve portion to slide into unwanted areas in the small intestine. The intestine sleeve portion can have a nutrient delivery layer located on an inside surface of the intestine sleeve portion which contain time-release mechanisms to slowly release nutrients to the small intestine over an extended period of time. The intestine sleeve portion can also adhere to the small intestine through the use of chitosan, alginate, and/or bioabsorbable polymers. Furthermore, the chitosan, alginate, and/or bioabsorbable polymers can be used instead of the intestine sleeve portion.

The intestine sleeve portion can also have a nutrient delivery cover located on an outside surface of the intestine sleeve portion. The nutrient delivery cover can be in direct contact with the small intestine and the time release nutrients to the small intestine over an extended period of time. In addition, the intestine sleeve portion can have an outer shell with a plurality of holes, and/or a semi-porous skin to allow nutrients to flow through the intestine sleeve portion to reach the small intestines.

The present invention can also include a cuff which can wrap around a portion or portions of the stomach and/or the small intestine. The cuff can be located outside the stomach and/or the small intestine while the endoluminal sleeve is located inside the stomach and/or the small intestine. The cuff and the endoluminal sleeve can be connected by a fastener to prevent the cuff and/or the endoluminal sleeve from sliding into an undesirable position within the stomach and/or the small intestine. The cuff can also include holes to accommodate blood vessels of the stomach, even when the cuff is wrapped around the stomach.

In one embodiment, the present invention is an apparatus for treating obesity and/or metabolic diseases, the apparatus including an endoluminal sleeve. The endoluminal sleeve can include a flexible core formed from a self-expanding memory material.

In another embodiment, the present invention is an apparatus for treating obesity including an endoluminal sleeve. The endoluminal sleeve can include an endoluminal sleeve formed from a mucosal adherent.

### BRIEF DESCRIPTION OF THE DRAWINGS

These and other embodiments of the invention will be discussed with reference to the following exemplary and non-limiting illustrations, in which like elements are numbered similarly, and where:

FIG. 1 is a view of an endoluminal sleeve positioned inside a stomach that has undergone a sleeve gastrectomy procedure according to an embodiment of the present invention;

FIG. 2 is a view of a stomach sleeve portion of an endoluminal sleeve positioned within a stomach according to an embodiment of the present invention;

FIG. 3 depicts an endoluminal valve in an endoluminal sleeve according to an embodiment of the present invention;

FIG. 4 is a overview of an outlet in an endoluminal valve according to an embodiment of the present invention;

FIG. 5 is a overview of an outlet in an endoluminal valve according to an alternate embodiment of the present invention;

FIG. 6 is a side view of an endoluminal valve according to an alternate embodiment of the present invention;

FIG. 7 is a close-up view of a portion of an endoluminal valve according to an alternate embodiment of the present invention;

FIG. 8 is a view of an antral sleeve portion of an endoluminal sleeve positioned within a pyloric antrum according to an embodiment of the present invention;

FIG. 9 is view of an intestine sleeve portion of an endoluminal sleeve positioned within a small intestine according to an embodiment of the present invention;

FIG. 10 is a view of an outer surface of an intestine sleeve portion of an endoluminal sleeve according to an embodiment of the present invention;

FIG. 11 is a side view of a nutrient delivery layer inside an intestine sleeve portion of an endoluminal sleeve according to an embodiment of the present invention;

FIG. 12 is a cross-sectional view of an endoluminal sleeve according to an embodiment of the present invention;

FIG. 13 is a side view of a nutrient delivery cover on an intestine sleeve portion of an endoluminal sleeve according to an embodiment of the present invention;

FIG. 14 is a view of an outer shell on an intestine sleeve portion of an endoluminal sleeve according to an embodiment of the present invention;

FIG. 15 depicts a physiological connection between stomach receptors and a brain according to an embodiment of the present invention;

FIG. 16 depicts an arrangement of wires for the mesh in the endoluminal sleeve according to an embodiment of the present invention;

FIG. 17 depicts an arrangement of wires for the mesh in the endoluminal sleeve according to an embodiment of the present invention;

FIG. 18 is a sectional view of an endoluminal sleeve and a cuff surrounding a stomach according to an embodiment of the present invention;

FIG. 19 depicts a cuff with a flap covering an upper portion of the stomach according to an embodiment of the present invention;

FIG. 20 depicts a cuff with a secured flap covering an upper portion of a stomach according to an embodiment of the present invention;

FIG. 21 depicts a cuff spanning a greater curvature and a lower curvature of a stomach according to an embodiment of the present invention;

FIG. 22 depicts a cuff covering a middle portion of a stomach according to an embodiment of the present invention;

FIG. 23 depicts a cuff covering a lower portion and a pyloric antrum of a stomach according to an embodiment of the present invention;

FIG. 24 depicts a cuff covering a pyloric antrum of a stomach and a portion of the lower intestine according to an embodiment of the present invention;

FIG. 25 depicts a cuff covering a portion of a stomach above a pyloric antrum according to an embodiment of the present invention;

FIG. 26 depicts a cuff covering a pyloric antrum of a stomach according to an embodiment of the present invention; and

FIG. 27 depicts an embodiment of the cuff according to an embodiment of the present invention.

### DETAILED DESCRIPTION

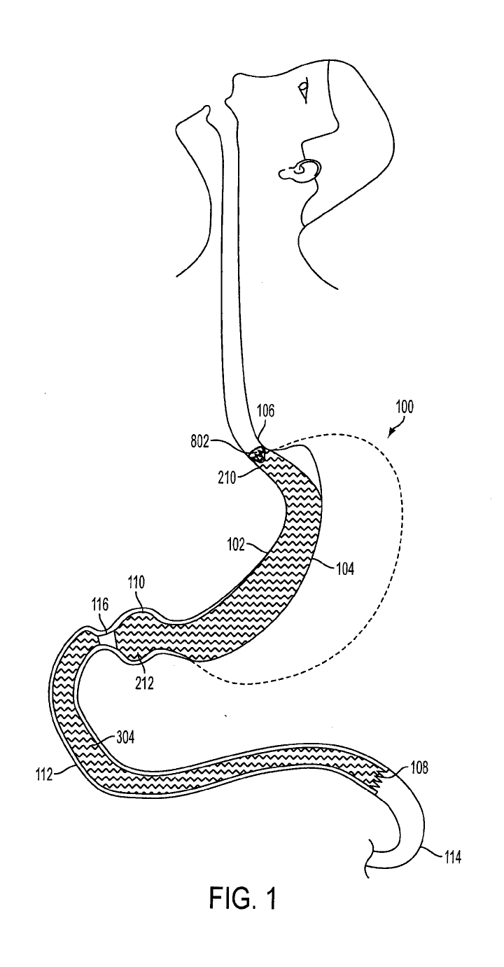

FIG. 1 is a view of an endoluminal sleeve 108 positioned inside a stomach 100 of a patient. The patient can, for example, be a patient that has undergone a sleeve gastrectomy procedure, be obese, have diabetes, and/or be at risk for diabetes. The patient can also be, for example a mammal, such as a human or an animal. The sleeve gastrectomy procedure is also known as a vertical gastrectomy, a greater curvature gastrectomy, a partial gastrectomy, a gastric reduction, a longitudinal gastrectomy, and/or a vertical gastroplasty. A significant portion of the stomach 100 is reduced following the sleeve gastrectomy procedure, so that the greater curvature 104 and the lower curvature 102 of the stomach 100 create a banana-shaped cavity. In one embodiment, an endoluminal sleeve 108 is positioned inside the stomach 100, and extends through the small intestine 112 and ends near the jejunum 114. The jejunum 114 is the middle section of the small intestine 112. The endoluminal sleeve 108 is held in place at the gastroesophogeal junction 106, as well at the pyloric antrum 110.

The endoluminal sleeve 108 can be placed, for example, within the stomach 100 by a user. The user can be, for example, a healthcare professional, such as a surgeon, a bariatric surgeon or a gastrointestinal specialist trained in endoscopic surgery procedures. In one embodiment, the endoluminal sleeve 108 can be positioned using a routine endoscopic procedure. In another embodiment, the endoluminal sleeve 108 can be placed within the stomach 100 using newer techniques, methods and procedures for endoscopic surgery. The endoluminal sleeve 108 can allow food to pass through the endoluminal sleeve 108 using, for example, Bernoulli's principle.

The present invention can be utilized in conjunction with the sleeve gastrectomy procedure, other bariatric procedures, and/or other post-bariatric procedures that provide stomach bypass, stomach reinforcement, tucking of the greater curvature of the stomach, and/or stomach restraining devices to prevent further expansion or re-expansion of the stomach 100.

In one embodiment, the endoluminal sleeve 108 is preferably formed from an alloy of nickel and titanium (“Nitinol”) or wire mesh. The wire mesh can be, for example, a self-expanding stainless steel wire mesh. The Nitinol or wire mesh can provide the endoluminal sleeve 108 with a self-expanding memory. For example, one of the unique characteristics of Nitinol is that it can self expand. This allows the endoluminal sleeve 108 to be crimped per a desired length, width, and volume based on the specific size required per the patient's stomach and intestinal dimensions. The endoluminal sleeve 108 can then be crimped into a sheath for endoscopic delivery. The endoluminal sleeve 108 regains its desired shape when deployed from the sheath. In one embodiment, the endoluminal sleeve 108 is covered with expanded Polytetrafluoroethylene (“ePTFE”), silicone, Dacron®, or any other elastomer material or thermoelastic elastomer material.

In one embodiment, the endoluminal sleeve 108 can be modular and can include, for example, an endoluminal valve 802, a stomach sleeve portion 210, an antral sleeve portion 212, and/or an intestine sleeve portion 304. For example, the stomach sleeve portion 210 can be in a top portion of the endoluminal sleeve 108, the antral sleeve portion 212 can be in a middle portion of the endoluminal sleeve 108, and the intestine sleeve portion 304 can be in a lower portion of the endoluminal sleeve 108.

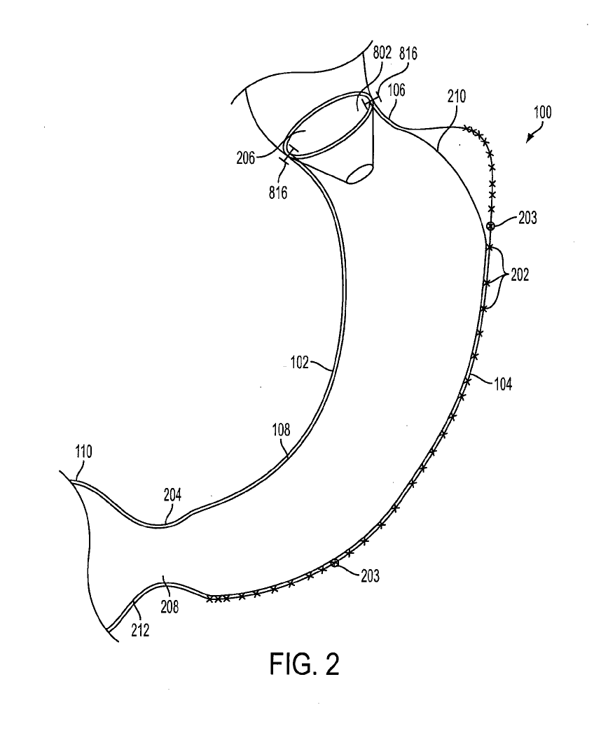

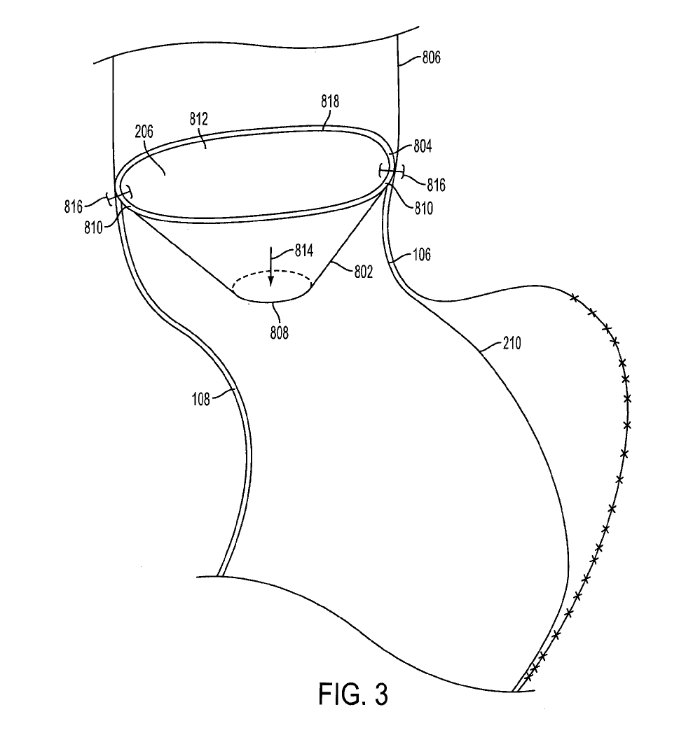

The endoluminal valve 802 and the stomach sleeve portion 210 of the endoluminal sleeve 108 can be seen, for example, in FIG. 2. The endoluminal valve 802 can commence usage of Bernoulli's principle in the endoluminal sleeve 108 to pass food through the endoluminal sleeve 108. The shape of the endoluminal valve 802 as seen in FIG. 2 can be beneficial in using Bernoulli's principle. The stomach sleeve portion 210 is positioned within the stomach 100 while the endoluminal valve is positioned at an inlet 206 to the stomach sleeve portion 210. As seen in FIG. 3, in one embodiment, the endoluminal valve 802 can include, for example, a ring 804, an inlet 812, and/or an outlet 808. The ring 804 can be formed, for example, from Nitinol or stainless steel wire mesh having a self-expanding memory.

In one embodiment, after endoscopic insertion into the body, the ring 802 expands to a size approximately equal to that of the esophagus 806, and forms a junction 810 with the esophagus 806. The junction 810 is snugly fitted to the esophagus 806 to prevent food from passing through the esophagus 806 without passing through the ring 802. The ring 802 allows a normal passage of food into the endoluminal valve 802 using the inlet 812. The endoluminal valve 802 provides a one-way, or unidirectional, passage of food from the outlet 808 into the endoluminal sleeve 108 as shown by the directional arrow 814. Food or stomach contents that are in the stomach sleeve portion 210 are prevented from passing through the outlet 808 of the endoluminal valve 802 and into the esophagus 806.

The endoluminal valve 802 can be positioned near a gastroesophogeal junction 106, and the junction 810 can assist in preventing gastroesophogeal reflux disease (“GERD”). Since acid tends to creep up the side of the stomach towards an esophagus 806, the junction 810 is a barrier to such acid movement.

During the sleeve gastrectomy procedure, the gastric sling fibers, which are short and long oblique muscular fibers in the stomach 100, are severed or cut out of the stomach 100. The sling fibers are critical in providing esophageal sphincter tone. With the sling fibers severed, the esophageal sphincter tone is laxed, and results in an increased tendency of GERD. The endoluminal valve 802 prevents relaxation of the esophageal sphincter, as the ring 804 provides a constant outward pressure on the walls of the esophagus 806. Thus, the endoluminal valve 802 assists in preventing esophageal sphincter laxation.

The endoluminal valve 802, due to its self-expanding nature, provides constant pressure on the walls of the esophagus 806. This outward pressure also holds the endoluminal valve 802 in place and prevents the endoluminal valve 802 from dislodging or sliding down into the stomach 100 and/or the small intestine 112. In one embodiment, the endoluminal valve 802 can also be secured in place through sutures 816 which can secure the endoluminal valve 802 and/or the stomach sleeve portion 210 to the stomach, the esophagus 806, and/or a junction between the stomach and the esophagus 806.

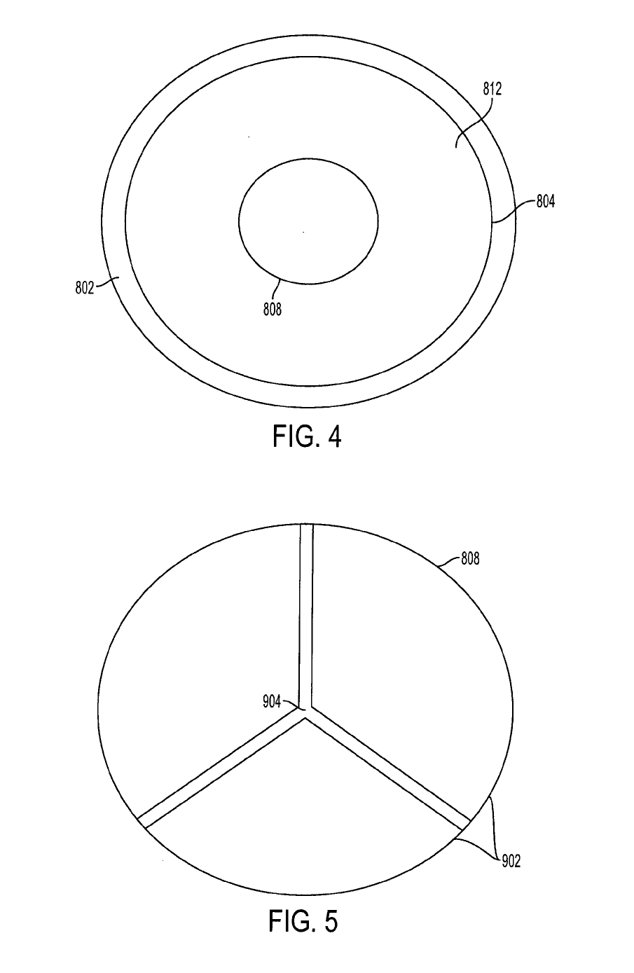

FIG. 4 depicts an embodiment of the outlet 808 of the endoluminal valve 802 as seen, for example, from the directional arrow 814. FIG. 5 depicts an alternate embodiment of the outlet 808 of the endoluminal valve 802 as seen, for example, from the directional arrow 814. In one embodiment, the outlet 808 includes multiple flexible flaps 902, which move downward when pressure from food is applied. When the flaps 902 move downward, food flows into the endoluminal sleeve 108 through the valve exit 904. The flaps 902 are designed to move downward upon the application of pressure, and then to return to a resting state. However, the flaps 902 cannot move in an upward direction beyond the resting state. Thus, food and stomach contents are prevented from passing through the outlet 808 and into the esophagus 806.

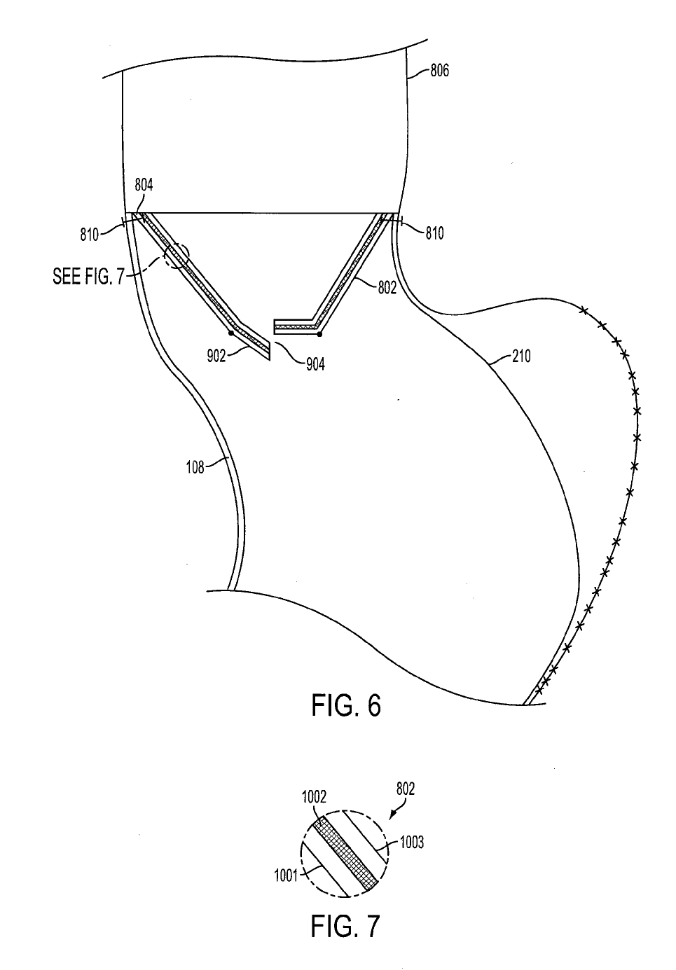

FIG. 6 is a side view of an embodiment of the endoluminal valve 802 with the outlet 808 partially open. When food enters the endoluminal valve 802 from the esophagus 806, the pressure is exerted on the flaps 902, causing the flaps 902 to move downward and open the valve exit 904. As seen in FIG. 7, the endoluminal valve 802 has an outer section 1001, a middle section 1002, and an inner section 1001. The food travels along the inner section 1003 of the endoluminal valve 802.

Referring back to FIG. 2, the stomach sleeve portion 210 includes the inlet 206 that is positioned near the gastroesophogeal junction 106. In another embodiment, the inlet 206 is positioned anywhere along the stomach 100 between the lower curvature 102 and the greater curvature 104. The stomach sleeve portion 210 is held in place by the outward pressure applied against the stomach walls by the self-expanding force of the Nitinol or the wire mesh. In one embodiment, the endoluminal sleeve 108 is not attached to the stomach walls by hooks, anchors, or other connecting means. Instead, the outward pressure applied by the endoluminal sleeve 108 on the stomach 100 prevents the endoluminal sleeve 108 from being dislodged from the stomach 100 or from sliding from the stomach 100 into undesired areas. The undesired areas can be, for example, areas in a digestive tract past the jejunum 114 such as other areas of the small intestine 112 or the large intestine. The endoluminal sleeve 108 fits snugly into the stomach 100.

The stomach sleeve portion 210 can also prevent leakage or ruptures of the stomach 100 following the sleeve gastrectomy procedure. During the sleeve gastrectomy procedure, a portion of the stomach cavity is removed and the greater curvature 104 of the stomach 100 is stapled, or alternatively, sutured, together, along a staple line 202. In some patients, the staple line 202 can leak or rupture due to pressure exerted from stomach contents or expansion of the stomach 100. Especially vulnerable points can include, for example, staple junction points 203. The staple junction points 203 are where multiple staple lines meet during the sleeve gastrectomy. This leakage can be life threatening, difficult to heal, and/or bleed. In one embodiment, the stomach sleeve portion 210 of the endoluminal sleeve 108 is positioned adjacent to the staple line 202 so that it reinforces the staple line 202, and prevents the stomach contents from coming into contact and potentially disrupting the staple line 202 and/or the staple junction points 203. Thus, leakage and/or bleeding is prevented as the contents entering the stomach 100 is routed into the inlet 206 and directly passed through to the lower intestine 112. Instead of or in addition to the stomach sleeve portion 210, mucosal adherent such as chitosan, alginates, and/or bioabsorbable polymers can be used, especially at vulnerable locations such as at the staple line 202 and/or the staple junction points 203. In addition any other polymers aside from bioabsorbable polymers may be used. The chitosan can be biodegradable allowing for the natural removal of the chitosan into the body. Thus, the chitosan can be a temporary or reversible solution instead of a permanent or irreversible solution.

In one embodiment, the stomach sleeve portion 210 overlaps with the antral sleeve portion 212 at a junction 208, positioned near the angular notch 204. The junction 208 is formed when complementary ends of the stomach sleeve portion 210 and the antral sleeve portion 212 expand and overlap with each other. In a preferred embodiment, there is about a 2 centimeter to 4 centimeter overlap between the complimentary ends.

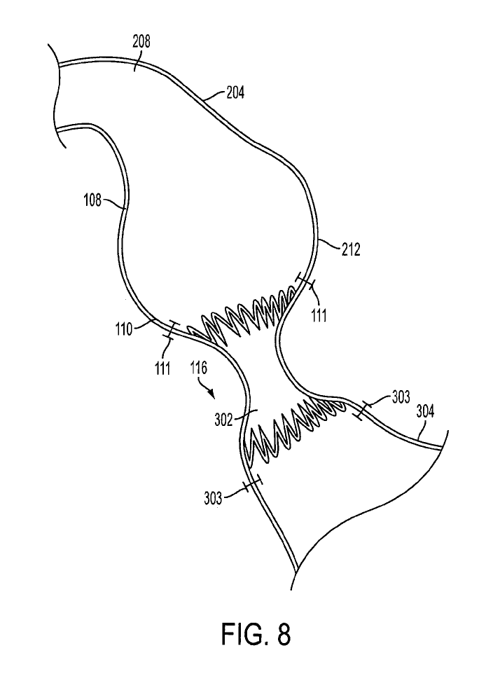

FIG. 8 depicts the antral sleeve portion 212 of the endoluminal sleeve 108. The antral sleeve portion 212 can be positioned within a pyloric antrum 110. In one embodiment, the antral sleeve portion 212 of the endoluminal sleeve 108 expands and is secured in place within the pyloric antrum 110. The pyloric antrum 110 is a portion of the stomach 100 where food and particles are collected and pumped into the lower intestine 112. The antral sleeve portion 212, due to its self-expanding nature, provides constant pressure on an inner wall of the pyloric antrum 110. The constant pressure by the antral sleeve portion 212 on the pyloric antrum 110 causes an indication to the stomach 108 that the pyloric antrum 110 is full. Due to the constant feeling of fullness, the pyloric antrum 110 continually attempts to pump out its contents. This continuous pumping actions leads to rapid gastric emptying of contents into the intestines. The physiological indications and reaction of pumping the stomach 100 is described in more detail herein. The antral sleeve portion 212 can be secured, for example, to the pyloric antrum 110 through the sutures 111.

In one embodiment, the antral sleeve portion 212 does not overlap with the intestine sleeve portion 304. Instead, a junction 302 in the endoluminal sleeve 108 is created near the pyloric sphincter 116. In one embodiment, the junction 302 does not contain any self-expanding material and instead is formed from non-self-expanding material. For example, the junction 302 can include an elastomeric cloth, preferably from ePTFE, silicone, Dacron®, or any other elastomer material or thermoelastic elastomer material. In another embodiment, the junction 302 is about 2 centimeter to about 4 centimeter in length and is connected between the ends of the intestine sleeve portion 304 and the antral sleeve portion 212. The junction 302 allows the pyloric sphincter 116 to function, as the junction 302 prevents the pyloric sphincter 116 from being constrained or restricted by pressure from a self-expanding material. For example, in one embodiment, the junction 302 allows the pyloric sphincter 116 to empty stomach contents. In another embodiment, the junction 302 can be devoid of material to maintain the pyloric sphincter in a properly functioning state to empty stomach contents.

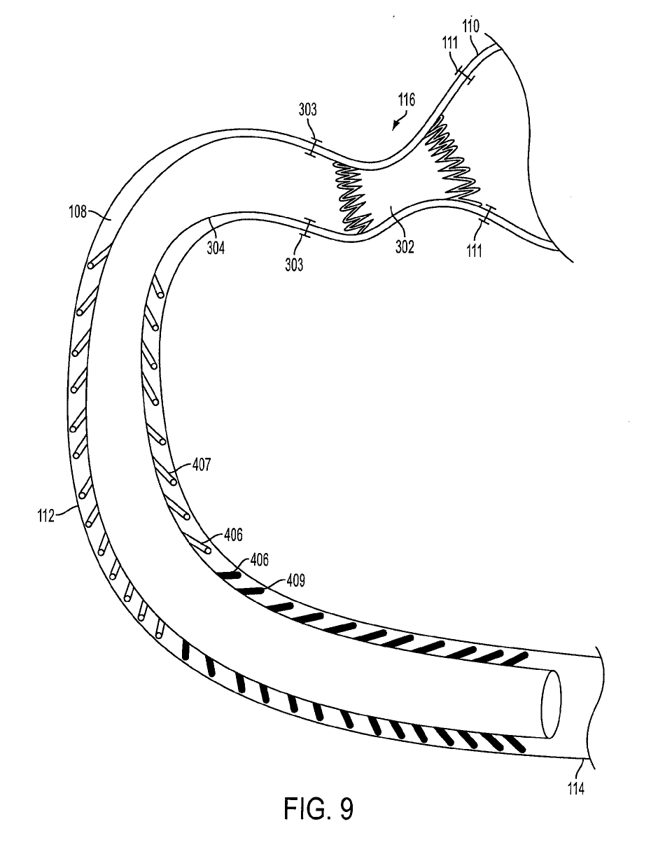

The intestine sleeve portion 304 of the endoluminal sleeve 108 can be seen, for example, in FIGS. 8 and 9. The intestine sleeve portion 304 can be positioned within the small intestine 112 and can be attached to the small intestine 112, for example, through the use of fasteners such as sutures 303. Multiple sutures 303 could be used, and the sutures 303 can be located at any portion of the small intestine 112 such as a top portion of the small intestine, a bottom portion of the small intestine, and/or the jejunum. The intestine sleeve portion 304 can also be secured to the small intestine 112 through the use of other fasteners instead of the sutures 303 or in combination with the sutures 303.

In one embodiment, the intestine sleeve portion 304 expands and is held in place within the small intestine 112, preferably between the pyloric sphincter 116 and the jejunum 114. In another embodiment, the intestine sleeve portion 304 includes protruding roots 406 that extend in an outward direction. The protruding roots 406 can be, for example, hollow portions 407, which allow nutrients to flow from the endoluminal sleeve 108 to the small intestine 112. The protruding roots 406 can each be, for example, of varying sizes and lengths, and can be positioned around the intestine sleeve portion 304 in a staggered or random pattern. In yet another embodiment, the roots 406 are solid portions 409 and do not allow nutrients to flow through to the small intestine 112. The solid portions 409 can be positioned in areas of the small intestine 112 where little nutrient absorption occurs. The solid portions 409 serve solely as a support mechanism for the intestine sleeve portion 304. The roots 406 can be uniform in size and length, and are positioned around the intestine sleeve portion 304 with equal space between each root. The roots 406 can also be positioned on an outside surface of the intestine sleeve portion 304. In one embodiment, the roots 406 can have a combination of hollow portions 407 and solid portions 409.

Furthermore, the roots 406 can provide support for the intestine sleeve portion 304, and/or prevent the intestine sleeve portion 304 from becoming dislodged. As the intestine sleeve portion 304 expands due to its self-expanding nature, the roots 406 come into contact with the inner wall of the small intestine 112. For example, the roots 406 can provide friction and stability, and prevent the intestine sleeve portion 304 from sliding down into lower portions of the small intestine 112 or even the large intestine.

In one embodiment, instead of or in addition to the roots 406, the intestine sleeve portion 304 can be coated with a biological material such as a mucosal adherent. The mucosal adherent can be, for example chitosan, alginate, and/or bioabsorbable polymers. The chitosan, alginate, and/or bioabsorbable polymers can function as an adhesive to prevent the intestine sleeve portion 304 from sliding down into lower portions of the small intestine 112 or even the large intestine. The intestine sleeve portion 304 can, for example, be dipped in chitosan, alginate, and/or bioabsorbable polymers. The intestine sleeve portion 304 can also, for example, be sprayed or coated with chitosan, alginate, and/or bioabsorbable polymers. The chitosan, alginate, and/or bioabsorbable polymers can also be applied instead to the small intestine 112 with the intestine sleeve portion 304 placed into the small intestine 112 after the small intestine 112 has been sprayed or coated with chitosan, alginate, and/or bioabsorbable polymers.

In another embodiment, the mucosal adherent, such as chitosan, alginate, and/or bioabsorbable polymers can be used instead of the intestine sleeve portion 304. The chitosan, alginate, and/or bioabsorbable polymers can be sprayed, coated, and/or adhered to the small intestine 112. The chitosan, alginate, and/or bioabsorbable polymers can allow food to pass quickly through the small intestine 112, functioning similar to the intestine sleeve portion 304. Furthermore, the chitosan, alginate, and/or bioabsorbable polymers may be porous and allow some nutrients to be absorbed by the small intestine 112. Since the chitosan, alginate, and/or bioabsorbable polymers can be biodegradable, the chitosan can be a temporary or reversible solution instead of a permanent or irreversible solution. Thus, a patient does not need to determine whether he wants to have surgery with permanent side effects. Instead, the patient can determine whether he wants to have surgery with temporary or very little side effects. Thus, the use of chitosan, alginate, and/or bioabsorbable polymers allows the procedure to be used for patients who are moderately obese since the procedure is not permanent and has reduced side effects. The chitosan, alginate, and/or bioabsorbable polymers can also allow the procedure to be used with patients who are moderately obese with a metabolic problem like diabetes since the procedure is not permanent and has reduced side effects.

In yet another embodiment, a modified intestinal scaffold can be used instead of the stomach sleeve portion 210, the antral sleeve portion 212, and/or the intestine sleeve portion 304. The modified intestinal scaffold can also be a biological material. The modified intestinal scaffold can be formed using xeno, hetero, or homo grafts. The modified intestinal scaffold can also be formed from stem cells. In still another embodiment, the stomach sleeve portion 210, the antral sleeve portion 212, and/or the intestine sleeve portion 304 can be formed from a biological material, derivative, and/or membrane that can be adhered to the stomach and/or the intestinal mucosa. In addition, the stomach sleeve portion 210, the antral sleeve portion 212, and/or the intestine sleeve portion 304 can be formed from biological material such as non-toxic animal products. The non-toxic animal products can be, for example, materials used in the food industry such as coatings for sausages. The non-toxic animal products can be porous or semi-porous to reduce absorption of fat and/or glucose.

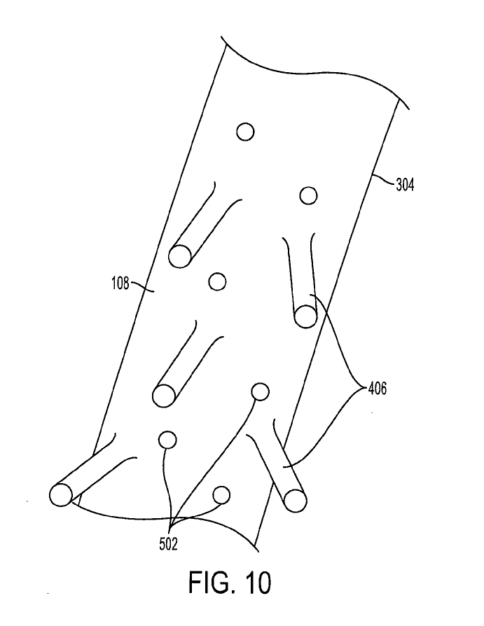

FIG. 10 is a view of an outer surface of the intestine sleeve portion 304 of the endoluminal sleeve 108. In an embodiment, the intestine sleeve portion 304 includes fenestrations 502, or holes, which allow nutrients to flow from the endoluminal sleeve 108 to the small intestine 112. The fenestrations 502 can be of different shapes and sizes, and can be positioned in a staggered or random pattern around the intestine sleeve portion 304. In another embodiment, the fenestrations 502 are uniform in size and length, and are positioned around the intestine sleeve portion 304 with equal space between each fenestration. The roots 406 and fenestrations 502 prevent a complete malabsorption of nutrients.

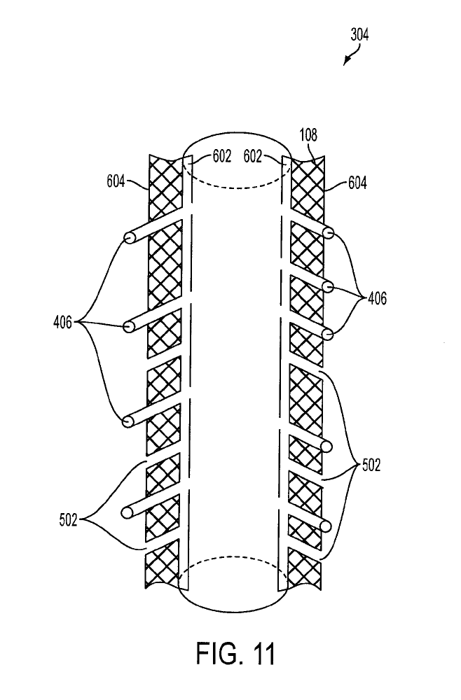

In one embodiment, the intestine sleeve portion 304 of the endoluminal sleeve 108 can include a nutrient delivery layer 602 and/or a flexible core layer 604 as shown in FIG. 6. The flexible core layer can be formed, for example, from Nitinol or stainless steel wire mesh having a self-expanding memory. In FIG. 11, the flexible core layer 604 covers the internal nutrient delivery layer 602. In one embodiment, the internal nutrient delivery layer 602 is positioned along an inner surface of the intestine sleeve portion 304. The nutrient delivery layer 602 contains nutrients, such as water, carbohydrates, proteins, fats, vitamins, and minerals, or any combination thereof. In a preferred embodiment, the nutrient delivery layer includes vitamins and minerals such as nitrogen, phosphorus, potassium, calcium, magnesium, sulfur, iron, copper, zinc, manganese, molybdenum, boron, Vitamin A, Vitamin B, Vitamin C, Vitamin D, Vitamin E, Vitamin K, and other fat-soluble and water-soluble vitamins, or any combination thereof.

In one embodiment, the nutrient delivery layer 602 has a time-release mechanism, allowing nutrients to slowly release into the body over an extended period of time. The nutrients are released and flow through the roots 406 and/or fenestrations 502 and into the small intestine 112 for absorption by the body.

In another embodiment, the nutrient delivery layer 602 is located only at select portions along the inner surface of the intestine sleeve portion 304. For example, the duodenum is largely responsible for the breakdown of food in the small intestine, using enzymes, and it is where most of the chemical digestion of food and absorption of nutrients takes place. The nutrient delivery layer 602 can be located at the duodenum, and other select areas along the intestine sleeve portion 304, so that the nutrient delivery is targeted to areas of the intestine that are responsible for nutrient absorption.

In another embodiment, the intestine sleeve portion 304 can be semi-porous and have, for example, a semi-porous membrane. The semi-porous membrane can allow nutrients from the nutrient delivery layer 602 to flow into the small intestine. In this embodiment, the roots 406 and/or the fenestrations 502 are not required, since the nutrients can flow directly through the semi-porous skin of the intestine sleeve portion 304.

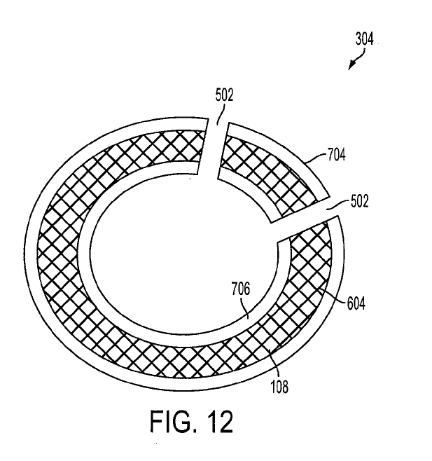

FIG. 12 is a cross-sectional view of an embodiment of the endoluminal sleeve 108. In one embodiment, the endoluminal sleeve 108 includes the flexible core layer 604. The flexible core layer 604 is surrounded by an outer layer 704 that is preferably made of ePTFE, silicone, Dacron® or any other elastomer or thermoelastic elastomer. The outer layer 704 allows movement of the flexible core layer 702, while protecting the stomach lining from friction or abrasion caused by the endoluminal sleeve 108. In one embodiment, the flexible core layer 604 surrounds an inner layer 706 that can be formed, for example, from ePTFE, silicone, Dacron® or any other elastomer material or thermoelastic elastomer material. The inner layer 706 allows food and other stomach contents to flow through the endoluminal sleeve 108 without being stuck or caught in the flexible core layer 604. The inner layer 706 facilitates the passage of food through the endoluminal sleeve 108.

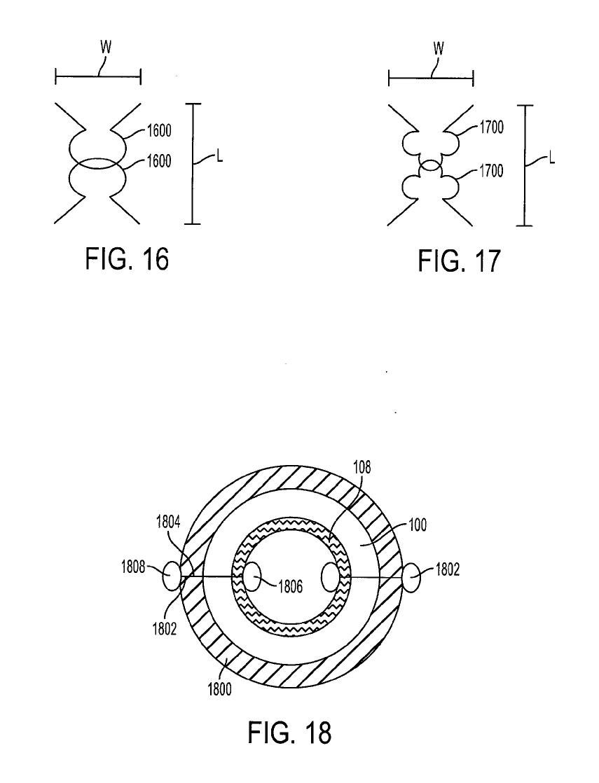

In one embodiment, the flexible core layer 604 is a mesh that can be formed, for example, from wiring patterns shown in FIGS. 16 and 17. In FIG. 16, the flexible core layer 604 can be formed from wires 1600 with a semi-circular end interlocked with each other. In FIG. 17, the flexible core layer 604 can be formed from wires 1700 with a club-shaped end interlocked with each other. The club-shaped end can allow greater flexibility for the flexible core layer 604. The club-shaped end can also allow the flexible core layer 604 to be stretched and moved while still retaining its mesh structure and without snagging or with substantially reduced snagging. When attached together, the club-shaped ends allow movement in all directions without snagging while still maintaining the hold between the two club-shaped ends.

The wire mesh can be formed from Teflon®, Dacron®, ePTFE, mesh, an elastic polymer (“elastomer”), a biodegradable and absorbable polymer or copolymer, and/or any other type of material suitable to reduce the expansion of stomach 100. The elastomer can be, for example, silicone, polypropylene, polyethylene terephthalate, polytetrafluoroethylene, polyaryletherketone, nylon, fluorinated ethylene propylene, or any combination thereof. Furthermore, the elastomer may be non-porous. Alternatively, the elastomer may be microporous or porous to allow for better expansibility and oxygenation and for tissue in-growth to better hold the flexible core layer 604 in place. The biodegradable and absorbable polymer or copolymer can be, for example, polyglycolic acid (PGA), polylactic acid (PLA), polycaprolactone, polyhydroxyalkanoate, various thermoplastic materials, or any combination thereof.

In both FIGS. 16 and 17, the wiring patterns from the wires 1600 and 1700 can have a width W and a length L. The width W and the length L can be variable depending on the location of the wiring patterns within the flexible core layer 604. In one embodiment, the width W and the length L is relatively smaller at an end of the flexible core layer and relatively larger at a middle portion of the flexible core layer. For example, near the pyloric antrum 110, the width W and the length L of the wirings 1600 and 1700 are relatively smaller than the width W and the length L of the wirings 1600 and 1700 near the middle of the stomach 100.

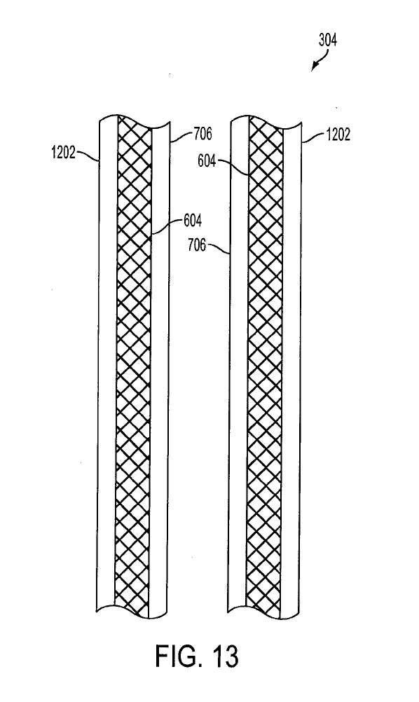

In another embodiment, the intestine sleeve portion 304 includes a nutrient delivery cover 1202, as seen in FIG. 13. The intestine sleeve portion 304 can also include the inner layer 706. The flexible core layer 604 includes a nutrient delivery cover 1202 that surrounds the flexible core layer 604. The nutrient delivery cover 1202 can function in a similar fashion the nutrient delivery layer 602 and deliver nutrients to the small intestine 112. However, the nutrient delivery cover 1202 is in direct contact with the small intestine 112, and allows for continuous delivery of nutrients to the body.

In an embodiment, portions of the nutrient delivery cover 1202 can include a higher concentration of nutrients than other portions. Thus, the nutrient delivery cover 1202 can provide selective delivery of a high concentration of nutrients to select portions of the intestine, such as, for example, the duodenum. In one embodiment, the intestine sleeve portion 304 uses the nutrient delivery cover 1202 instead of the fenestrations 502 and/or the roots 406.

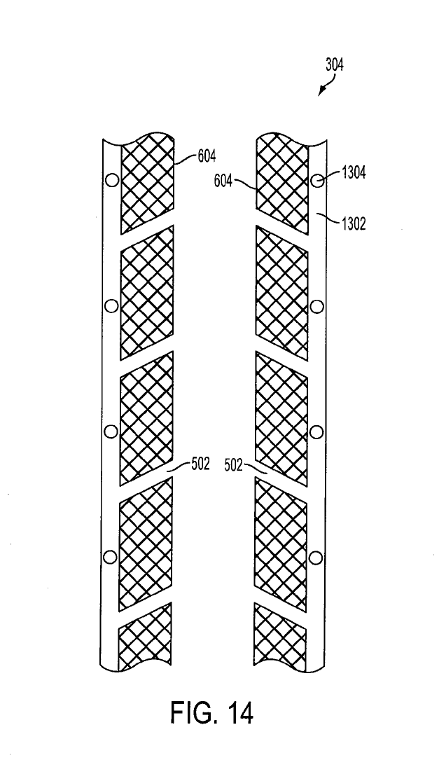

In another embodiment, as shown in FIG. 14, the intestinal sleeve portion 304 includes an outer shell 1302 and fenestrations 502. The outer shell 1302 includes a plurality of holes 1304 and is connected to the fenestrations 502. Thus, nutrients from the food passing through the intestinal sleeve portion 304 can pass through the fenestrations 502 and the plurality of holes 1304 into the small intestine 112. This allows for the delivery of nutrients to the body and reduces the malabsorption of nutrients.

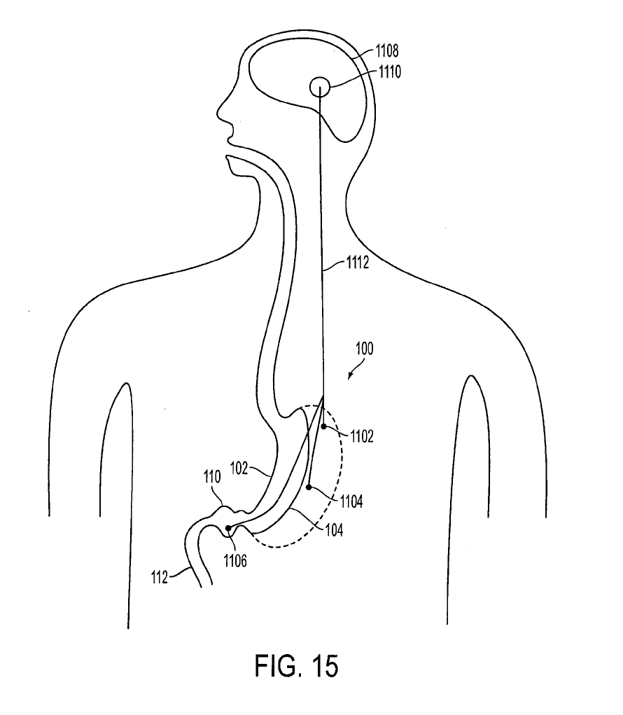

FIG. 15 is a view of a physiological connection between stomach receptors and a brain. After a sleeve gastrectomy procedure, many of the cardia stretch receptors 1102 and the stomach body stretch receptors 1104 are removed. However, the pyloric antrum 110 is not modified by the sleeve gastrectomy procedure, and the antral stretch receptors 1106 in the pyloric antrum 110 remain intact. Upon being filled with food and stomach contents, the pyloric antrum 110 expands, and the stretch receptors 1106 in the pyloric antrum 114 send neurological signals to the hypothalamus 1110 in the brain 1108, indicating the stomach is full. Upon receipt of these signals, the hypothalamus 1110 sends a signal via the afferent vagal nerve 1112 to the pyloric antrum 110 to pump out the food into the intestines.

The antral sleeve portion 212 provides a constant pressure around the pyloric antrum 110, so that when pyloric antrum 110 even slightly expands, the antral stretch receptors 1106 are constrained from further expansion. Upon being prevented from further expansion, the antral stretch receptors 1106 send a signal to the brain 1108, and in turn, the pyloric antrum 110 is caused to pump out food contents. The combination of the antral sleeve portion 212 of the endoluminal sleeve 108 and the antral stretch receptors 1106 create an equal and opposite reaction, causing a continual gastric emptying by the pyloric antrum 110. Thus, the present invention takes advantage of the antral stretch receptors 1106 that remain after the sleeve gastrectomy procedure in order to provide an indication of fullness to the brain 1108 and cause rapid and early gastric emptying.

In one embodiment, a cuff can be used in conjunction with the endoluminal sleeve 108. As seen in FIG. 18, a cuff 1800 surrounds an exterior of the stomach 100 while the endoluminal sleeve 108 is placed in an interior of the stomach 100. The cuff 1800 can prevent the stomach 100 from expanding, or reduce the expansion of the stomach 100. While wrapping the cuff 1800 over the stomach 100, a balloon, such as an endoscopic balloon, can be inflated inside the stomach 100 to help guide the cuff 1800 and/or prevent the cuff 1800 from being too tight or too loose around the stomach 100. The cuff 1800 can be secured to the endoluminal sleeve 108 through one or more fasteners 1802. The fastener 1802 can include an elongated portion 1804 and anchors 1806 and 1808. The anchors 1806 and 1808 have diameters larger than a diameter of the elongated portion 1804. The anchors 1806 and 1808 prevent the fastener 1802 from becoming dislodged and also prevent the cuff 1800 and the endoluminal sleeve 108 from substantially slipping or moving around.

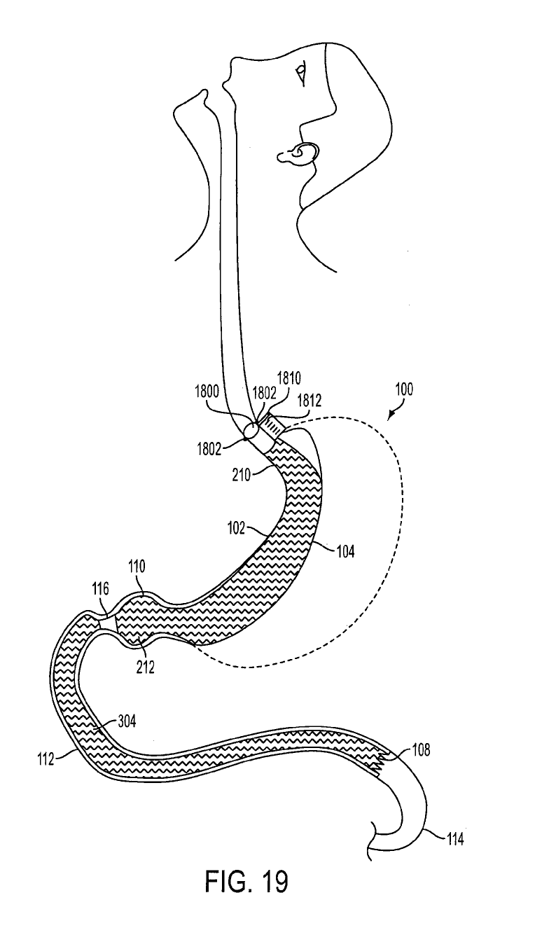

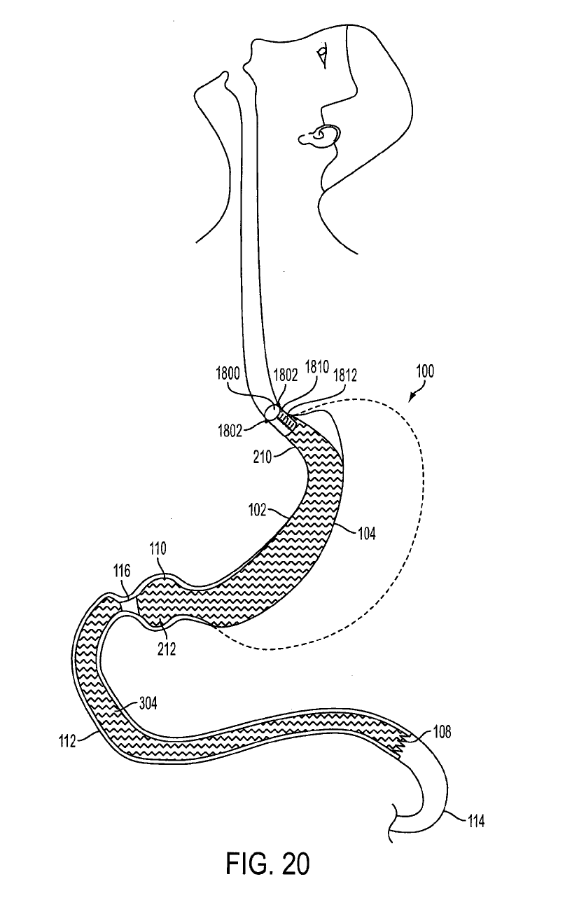

As can be seen in FIG. 19, the cuff 1800 can be placed in a top portion of the stomach 100. The cuff 1800 can be wrapped around the stomach 100 forming two flaps 1810. The flaps 1810 can be stapled together at staple line 1812. As seen in FIG. 20, the cuff 1800 can also have the flaps 1810 secured to the cuff 1800 through the sutures 1812. This can reduce the intrusion of the flaps 1810 and/or reduce the likelihood of the flaps 1810 being damaged, or the flaps 1810 causing damage. In FIG. 19, the cuff 1800 can be, for example, a proximal cuff.

The cuff 1800 can be formed from Teflon®, Dacron®, ePTFE, mesh, an elastic polymer (“elastomer”), a biodegradable and absorbable polymer or copolymer, and/or any other type of material suitable to reduce the expansion of stomach 100. The elastomer can be, for example, silicone, polypropylene, polyethylene terephthalate, polytetrafluoroethylene, polyaryletherketone, nylon, fluorinated ethylene propylene, or any combination thereof. Furthermore, the elastomer may be non-porous. Alternatively, the elastomer may be microporous or porous to allow for better expansibility and oxygenation and for tissue in-growth to better hold the cuff 1800 in place. The biodegradable and absorbable polymer or copolymer can be, for example, polyglycolic acid (PGA), polylactic acid (PLA), polycaprolactone, polyhydroxyalkanoate, various thermoplastic materials, or any combination thereof. The cuff 1800 can be located at any location along the stomach 100 and/or the small intestine 112. The cuff 1800 can also cover any portion of the stomach 100 and/or the small intestine 112.

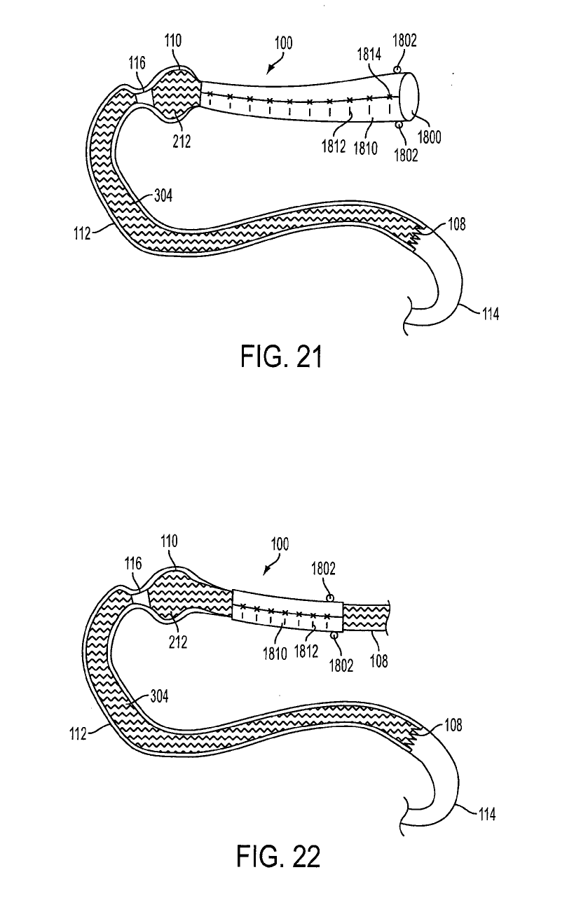

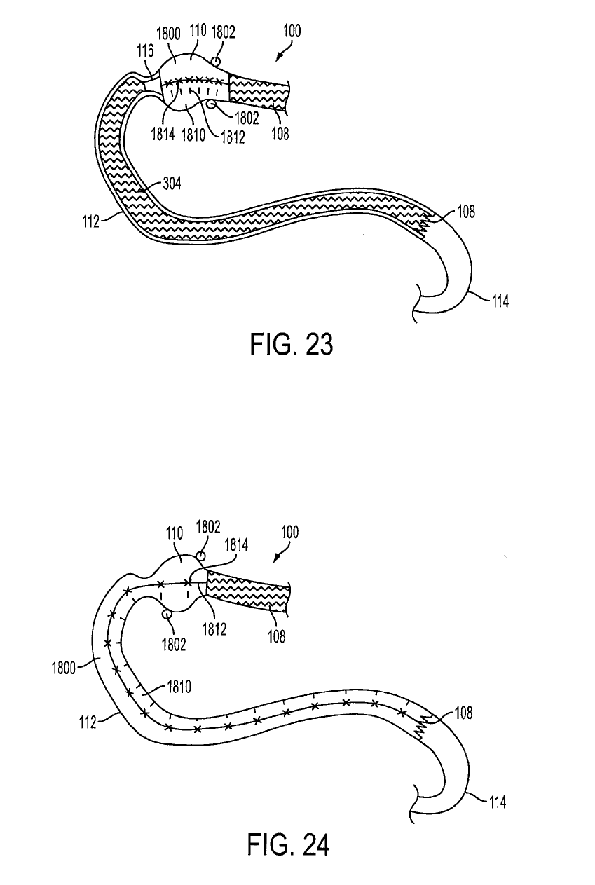

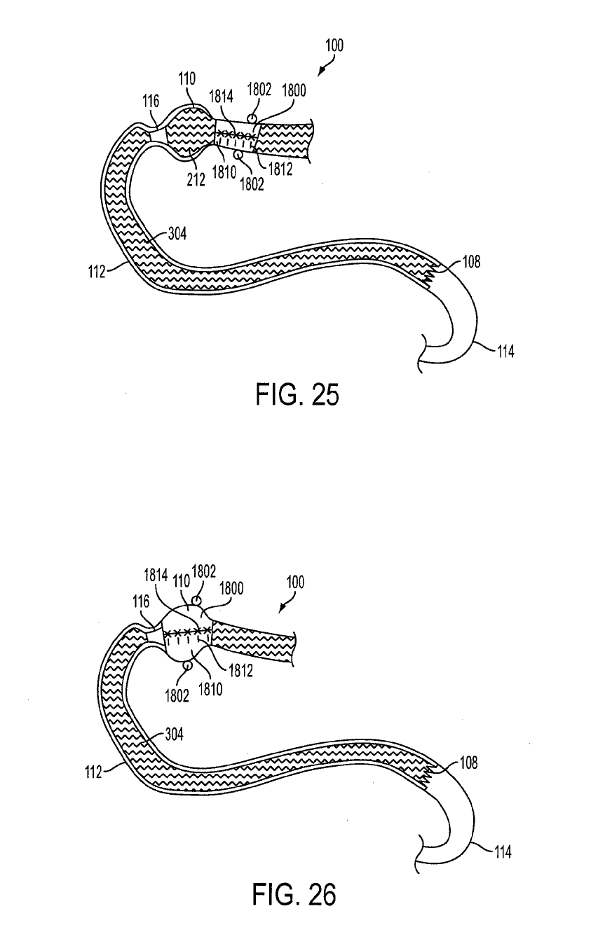

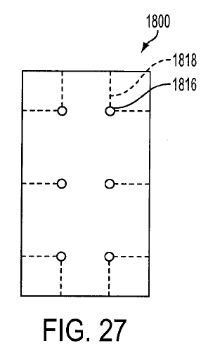

For example in FIG. 21, the cuff 1800 can cover the entire body of the stomach 100, including the neck of the stomach 100, before the pyloric antrum 110. In FIG. 22, the cuff 1800 can cover a middle portion of the stomach 100. As can be seen in FIG. 22, the cuff 1800 does not cover the neck of the stomach 100 or the pyloric antrum 110. In FIG. 23, the cuff 1800 covers the pyloric antrum 110 and also a lower portion of the stomach 100 above the pyloric antrum 110. In FIG. 24, the cuff 1800 covers the pyloric antrum 110 and a portion of the small intestine 112 to the end of the duodenum or the first part of the jejunum. In FIG. 25, the cuff 1800 covers a region of the stomach 100 above the pyloric antrum 110 and can be, for example, a distal cuff. In FIG. 26, the cuff 1800 covers the pyloric antrum 110.

In one embodiment, the cuff 1800 can have a length, for example, of about 2-25 cm. For example, in FIGS. 19 and 20, the cuff 1800 can be between about 2-6 cm long or about 2-4 cm long. However, the cuff 1800 can also be of any length. In extending the length of the cuff 1800, a portion of the cuff 1800 can have a formation shown in FIG. 27. In FIG. 27, the cuff 1800 includes holes 1816 and perforations 1818. The holes 1816 can be, for example, apertures. Thus, when the cuff 1800 is placed over the stomach 100, the holes 1816 can allow the cuff 1800 to cover portions of the stomach 100 where blood vessels connect to the stomach 100. The holes 1816 allow the blood vessels to pass through the cuff 1800 to the stomach 100.

To allow the blood vessels to enter and be located within the holes 1816, a doctor or person placing the cuff 1800 would only need to tear the perforations 1818. The perforations 1818 that are torn can be selected by the doctor or person placing the cuff 1800. In one embodiment, the perforations 1818 that are torn are those which provide the blood vessels the most convenient access to the holes 1816. With the holes 1816, the cuff 1800 can also have an extended length beyond about 2-25 cm since the blood vessels will not prevent the cuff 1800 from wrapping around the stomach 100. In one embodiment, the holes 1816 can also be punch-through holes so that only the holes 1816 which are going to be used need to be punched through.

While the principles of the disclosure have been illustrated in relation to the exemplary embodiments shown herein, the principles of the disclosure are not limited thereto and include any modification, variation or permutation thereof.

What is claimed is:

1. An apparatus for treating obesity or diabetes comprising:
an endoluminal sleeve including a flexible core formed from a self-expanding memory material.

2. The apparatus of claim 1 wherein the self-expanding memory material includes at least one of a Nitinol wire mesh or a stainless steel wire mesh.

3. The apparatus of claim 1 wherein the endoluminal sleeve includes an outer layer formed on top of the flexible core and formed from an elastomer material or a thermoelastic elastomer material.

4. The apparatus of claim 1 wherein the endoluminal sleeve includes an intestine sleeve portion.

5. The apparatus of claim 4 wherein the intestine sleeve portion includes roots.

6. The apparatus of claim 5 wherein the roots are hollow.

7. The apparatus of claim 6 wherein the roots are solid.

8. The apparatus of claim 4 wherein the intestine sleeve portion includes fenestrations.

9. The apparatus of claim 4 wherein the intestine sleeve portion includes a nutrient delivery layer located on an inner surface of the intestine sleeve portion.

10. The apparatus of claim 4 wherein the intestine sleeve portion includes a nutrient delivery cover located on an outside surface of the intestine sleeve portion.

11. The apparatus of claim 4 wherein the intestine sleeve portion is semi-porous.

12. The apparatus of claim 4 wherein the endoluminal sleeve includes an antral sleeve portion connected to the intestine sleeve portion.

13. The apparatus of claim 12 wherein the endoluminal sleeve includes a stomach sleeve portion connected to the antral sleeve portion.

14. The apparatus of claim 13 wherein the endoluminal sleeve further includes a junction formed from a non-self-expanding material, the junction connecting the antral sleeve portion and the intestine sleeve portion.

15. The apparatus of claim 14 wherein the non-self-expanding material is an elastomeric cloth.

16. The apparatus of claim 1 wherein the endoluminal sleeve includes a stomach sleeve portion, the stomach sleeve portion including an inlet portion.

17. The apparatus of claim 16 further comprising an endoluminal valve located in the inlet portion of the stomach sleeve, the endoluminal valve including a ring formed from a self-expanding memory material, and an outlet.

18. The apparatus of claim 17 wherein the endoluminal valve includes a plurality of flaps located at the outlet of the endoluminal valve allowing a unidirectional flow of material through the outlet of the endoluminal valve.

19. The apparatus of claim 1 wherein the endoluminal sleeve is coated with a mucosal adherent.

20. The apparatus of claim 19 wherein the mucosal adherent is chitosan.

21. The apparatus of claim 1 further comprising a cuff configured to surround a portion of a stomach, wherein the endoluminal sleeve is configured to be located within the stomach and the cuff is configured to be located outside the stomach.

22. The apparatus of claim 21 wherein the cuff is connected to the endoluminal sleeve.

23. The apparatus of claim 21 wherein the cuff is also configured to surround a portion of a small intestine.

24. The apparatus of claim 21 wherein a portion of the cuff forms apertures allowing a blood vessel of the stomach to pass through the cuff.

25. An apparatus for treating obesity or diabetes comprising:
an endoluminal sleeve formed from a biological material.

26. The apparatus of claim 25 wherein the biological material is a mucosal adherent formed from chitosan, alginate, or a bioabsorbable polymer.

27. The apparatus of claim 25 wherein the biological material is a scaffolding formed from stem cells.

28. The apparatus of claim 25 wherein the biological material is formed from non-toxic animal products which reduce absorption of glucose and fat by a stomach or a small intestine.

29. An endoluminal sleeve comprising:
a stomach sleeve portion having a top portion configured to be positioned adjacent to an esophagus, and a bottom portion, the stomach sleeve portion defining a passageway for food; and
an intestine sleeve portion configured to be located in a small intestine, having a plurality of fenestrations and a plurality of roots, the plurality of roots protruding from the intestine sleeve portion.

30. The endoluminal sleeve of claim 29 wherein the plurality of roots include a plurality of hollow roots and a plurality of solid roots.

31. The endoluminal sleeve of claim 29 further comprising an antral sleeve portion configured to be located adjacent a pyloric antrum, and between the stomach sleeve portion and the intestine sleeve portion.

32. The endoluminal sleeve of claim 31 further comprising a junction formed from a non-self-expanding material and configured to be located adjacent the pyloric sphincter, the junction connecting the antral sleeve portion and the intestine sleeve portion.

33. The endoluminal sleeve of claim 29 further comprising:
a first wire mesh covering the stomach sleeve portion, including wires formed in a club shape; and
a second wire mesh covering the intestine sleeve portion, including wires formed in a club shape.

34. The endoluminal sleeve of claim 33 wherein the first wire mesh and the second wire mesh include self-expanding material.

35. The endoluminal sleeve of claim 29 further comprising a cuff attached to the top portion of the stomach sleeve portion.

36. The endoluminal sleeve of claim 29 wherein the intestine sleeve portion is configured to be located adjacent a jejunum.

37. The endoluminal sleeve of claim 29 wherein the intestine sleeve portion is sutured to the small intestine.

38. The endoluminal sleeve of claim 29 wherein the stomach sleeve portion is sutured to the esophagus, a stomach, or a junction between the esophagus and the stomach.
